(19) United States
(12) Patent Application Publication
(10) Pub. No.: US 2025/0345788 A1
(43) Pub. Date: Nov. 13, 2025

(54) BIODEGRADABLE, WATER-DISSOLVABLE AND MOULDABLE CASSETTE FOR LATERAL FLOW ASSAY DEVICE, AND LATERAL FLOW ASSAY DEVICE COMPRISING SAID CASSETTE

(71) Applicant: Okos Diagnostics B.V., Leiden (NL)

(72) Inventors: Sander Julian Brus, Leiden (NL); Luis Fernando Sánchez Barrios, Leiden (NL)

(73) Assignee: Okos Diagnostics B.V., Leiden (NL)

(21) Appl. No.: 18/862,564

(22) PCT Filed: May 4, 2023
(86) PCT No.: PCT/NL2023/050240

§ 371 Date: Nov. 4, 2024

(30) Foreign Application Priority Data
May 5, 2022 (NL) 2031786

(51) Int. Cl. B01L 3/00; B01L 9/00; G01N 33/543

(52) U.S. Cl. CPC B01L 3/5023; B01L 9/52; G01N 33/54388; B01L 2200/12; B01L 2300/021; B01L 2300/0825; B01L 2400/0406

(57) ABSTRACT

A biodegradable and mouldable cassette for lateral flow assay device, and a lateral flow assay device comprising said cassette, and a kit comprising such a lateral flow assay device, for in vitro diagnostics of a biological sample comprising a diagnostic test strip inserted into the cassette. The cassette comprises a first cassette part and a second cassette part coupled to each other and defining a test chamber there between containing the diagnostic test strip. The cassette is made of a biodegradable material and at least one of the first cassette part and second cassette part is made of injection mouldable material, in particular gelatin or a gelatin composition.

### TECHNOLOGICAL FIELD

The invention relates to the field of lateral flow assay devices. More in particular, the invention relates to a cassette for a lateral flow assay device, and to the manufacturing of said cassette, and to a kit comprising such a lateral flow assay device.

### BACKGROUND

Cassettes are widely used, for example, in lateral flow assay devices or tests, for in vitro diagnosis of various biological samples. Known examples are pregnancy tests, malaria tests, HIV tests and tests for a coronavirus, particularly SARS-CoV-2. In general lateral flow assay devices are useful for quickly and/or accurately detecting the presence or absence of a predetermined test compound, including cells, antigens, and drug compounds and their metabolites in a sample. Known lateral flow assay devices to this end utilize a diagnostic test strip, usually a paper strip made of cellulose, comprising indicator means for the predetermined test compound and the test strip contained in a cassette that serves to protect the test strip and maintain the test strip in intact form prior to use. Most of the available lateral flow assay devices have the cassette made from plastics (such as made from fossil oil-based polymers) which allows for a quick and relative easy low cost production. However with the very high number of lateral flow assay tests being produced and sold each year globally these plastic cassettes form an undesirable amount of non-recyclable plastic waste. This is even more of a problem in view of the demand for point of care testing growing each year. As a consequence lateral flow assay devices have been developed that comprise a biodegradable cassette. A known example is a pregnancy test comprising of a housing made of cellulose.

Although the known biodegradable cassette offers a more environmental friendly solution as compared to the available plastic examples, there is a further need for an improved cassette for e.g. application in a lateral flow assay device.

### SUMMARY

A first aspect of the invention relates to a cassette suitable for application in a lateral flow assay device for in vitro diagnostics of a biological sample said lateral flow assay device comprising at least one diagnostic test strip, preferably one, two, three or four diagnostic test strip(s), inserted into said cassette, the cassette comprising a first cassette part and a second cassette part configured to be coupled to each other and defining after coupling at least one test chamber there between configured to contain the at least one diagnostic test strip, wherein the cassette is made of a biodegradable material and wherein at least one of the first cassette part and second cassette part is made of mouldable material, wherein the mouldable material is gelatin or a gelatin based biodegradable material. Importantly, the cassette provides for a universal housing for application in e.g. lateral flow assay devices. That is to say, the cassette is applicable in virtually any and all known lateral flow assay devices or tests, for in vitro diagnosis of various biological samples, e.g.

currently in use. This provides the benefit of, amongst others, suitability for mass production, optimized usage of resources and manufacturing facilities, optimized speed of production, etc., etc.

An embodiment is the cassette, wherein the gelatin or the gelatin based biodegradable material is water-dissolvable gelatin or water-dissolvable gelatin based biodegradable material.

An embodiment is the cassette, wherein the gelatin based biodegradable material comprises a biodegradable and water-dissolvable plasticizer such as any one or more of glycerol, sorbitol and propylene glycol, preferably glycerol. For example, the gelatin based biodegradable material comprises 2-50% water-dissolvable plasticizer such as glycerol based on the total weight of the material, and for example 50-98% gelatin based on the total weight of the material.

An embodiment is the cassette, wherein the first cassette part and the second cassette part are made of mouldable, preferably injection mouldable gelatin or a gelatin based biodegradable material.

An embodiment is the cassette, wherein the first cassette part and the second cassette part are made of the same mouldable, preferably injection mouldable gelatin or a gelatin based biodegradable material.

An embodiment is the cassette, wherein at least one of the first cassette part and second cassette part is moulded, preferably injection moulded in one piece.

An embodiment is the cassette, wherein the first cassette part and the second cassette part are each moulded, preferably injection moulded in one piece.

An embodiment is the cassette, wherein the first cassette part and the second cassette part are configured to be coupled to each other with a manually releasable connection.

An embodiment is the cassette, wherein the first cassette part is a lower cassette part and the second cassette part is an opposite upper cassette part configured to be coupled to each other such that, after coupling, at least one test chamber between the lower cassette part and the upper cassette part is defined, said at least one test chamber configured to contain the at least one diagnostic test strip.

An embodiment is the cassette, wherein the first cassette part is an inner cassette part and the second cassette part is an outer cassette part configured to slidably receive the inner cassette part, the inner cassette part and the outer cassette part configured to be slidably coupled to each other such that, after coupling, at least one test chamber between the inner cassette part and the outer cassette part is defined, said at least one test chamber configured to contain the at least one diagnostic test strip.

A second aspect of the invention relates to a lateral flow assay kit comprising a lateral flow assay device comprising the cassette according to the invention and at least one diagnostic test strip inserted into said cassette, and at least one of the group consisting of at least one replacement diagnostic test strip, a sample taker such as a swab, a sample holder such as a tube, and a waste container such as a zip-lock bag, wherein optionally at least one of the at least one diagnostic test strip and the group consisting of at least one replacement diagnostic test strip, a sample taker such as a swab, a sample holder such as a tube, and a waste container such as a zip-lock bag is made of a biodegradable material.

A third aspect of the invention relates to a method of manufacturing the cassette according to the invention, comprising or consisting of the steps:
(i) providing molten gelatin or the gelatin based biodegradable material;
(ii) providing the first cassette part and the second cassette part by moulding, preferably injection moulding of the molten gelatin or the gelatin based biodegradable material of step (i); optionally
(iii) coupling the first cassette part and the second cassette part provided in step (ii), such that at least one test chamber there between is defined configured to contain at least one diagnostic test strip; and optionally
(iv) providing, for example by printing, an exposed outer surface (95′) of the second cassette part (4, 90B, 400) with a mark, text, figure, label and/or code (91), such as a QR code,
   therewith providing the cassette.

A QR code could be printed on one of the two cassette parts, for example for the purpose of data storage or in order to share personalised results. For example, a trademark, copyright sign, reference number or label, indication of the analyte, etc., can be stamped or printed on the outer surface of the first or second cassette part.

### SHORT DESCRIPTION OF THE DRAWINGS

FIG. 1A shows an exploded view of an embodiment of a cassette for a lateral flow assay device in accordance with a first aspect herein.

FIG. 1B shows an exploded view of an embodiment of a lateral flow assay device in accordance with an embodiment herein, the device comprising the cassette of FIG. 1A.

FIG. 1C shows an exploded view of an alternative embodiment of a cassette for a lateral flow assay device in accordance with a first aspect herein.

FIG. 1D shows an exploded view of an alternative embodiment of a lateral flow assay device in accordance with an embodiment herein, the device comprising the cassette of FIG. 1C.

FIG. 1E shows an exploded view of an alternative embodiment of a cassette for a lateral flow assay device in accordance with an alternative aspect herein.

FIG. 1F shows an exploded view of an alternative embodiment of a lateral flow assay device in accordance with an embodiment herein, the device comprising the cassette of FIG. 1E.

FIG. 1G shows a view of the lateral flow assay device of FIG. 1F.

FIG. 1H shows an exploded view of an embodiment of a cassette for a lateral flow assay device comprising three test chambers in accordance with an aspect herein.

FIG. 1I shows an exploded view of an embodiment of a lateral flow assay device in accordance with an embodiment herein, the device comprising the cassette of FIG. 1H.

### DETAILED DESCRIPTION

It is an aim to provide an improved cassette for use as part of a biodegradable lateral flow assay device. In particular it is an aim to provide a biodegradable cassette for a lateral flow assay device that is reliable and/or convenient to produce. Another aim is to provide an improved lateral flow assay kit, in particular an improved lateral low assay kit comprising in addition to an improved biodegradable cassette for the lateral flow assay device one or more biodegradable kit parts such as a biodegradable swab and/or a biodegradable buffer liquid container.

Accordingly, in an aspect herein there is provided a cassette for application in a lateral flow assay device for in vitro diagnostics of a biological sample said lateral flow assay device comprising at least one diagnostic test strip, preferably one, two, three or four diagnostic test strip(s) (although more than four strips such as five-fifteen strips, for example six, seven, eight, nine, ten, eleven or twelve strips, is equally suitable), inserted into said cassette, or a lateral flow assay device comprising said cassette for in vitro diagnostics of a biological sample comprising at least one diagnostic test strip, such as one, two, three, four or five, preferably one, two or three such as one or three diagnostic test strip(s), inserted into the cassette. The cassette comprising in one embodiment a lower cassette part and an opposite upper cassette part coupled to each other and defining at least one test chamber there between containing the at least one diagnostic test strip (such as one or three test chambers there between containing one or three diagnostic test strip(s), respectively). The cassette comprising in one embodiment an inner cassette part and an outer cassette part configured to slidably receive the inner cassette part, the inner cassette part and the outer cassette part configured to be slidably coupled to each other, and after coupling defining at least one test chamber there between containing the at least one diagnostic test strip (such as one or three test chambers there between containing one or three diagnostic test strip(s), respectively). The cassette is made of a biodegradable material and at least one of the first cassette part and second cassette part is made of mouldable material, preferably injection mouldable material, and/or made of 3D printable curable material. The at least one of the first cassette part and second cassette part of the cassette made from a biodegradable and mouldable and/or 3D printable curable material enables a convenient production of the lateral flow assay device using moulding such as injection moulding or using 3D printing while being environmental friendly in view of the biodegradability of the cassette. Preferably the mouldable material and/or 3D printable curable material is curable from a liquid phase into a solid phase relatively easily without requiring elaborate and/or costly processes. Optionally, the mouldable, preferably injection mouldable material and/or 3D printable curable material can be cured from a liquid phase into a solid phase by changing the temperature of the material and/or by drying the material. Optionally the mouldable, preferably injection mouldable material and/or 3D printable curable material is provided in a liquid phase, i.e. as a liquid, for moulding and/or 3D printing of the material and is dried by exposure to air after moulding or printing to provide the mouldable and/or 3D printable curable material in cured form to make the at least one of the first cassette part and second cassette part of the lateral flow assay device in solid and durable useable form. Preferably the (injection) mouldable material and/or 3D printable curable material is cured by exposure to air at room temperature, i.e. approximately between 17-22° C. In embodiments, the material is mouldable material, preferably injection mouldable material. Such material can also be suitable for 3D printing. Moulded material can be cured as described after moulding, like 3D printed material. It is preferred that the cassette is made of a water dissolvable material which is preferably also a biodegradable material and at least one of the first cassette part and second cassette part is made of said water dissolvable material, which is preferably also biodegradable material. Preferably, the cassette is made of a water dissolvable material which is preferably also a biodegradable material and at least one of the first cassette part and second cassette part is made of said water dissolvable material, which is preferably also biodegradable material, wherein said material preferably also is a mouldable material, preferably injection mouldable material, and/or 3D printable curable material. The material is preferably dissolvable in water wherein the water is at ambient temperature such as room temperature, for example a temperature selected from the range 15° C.-30° C., such as 18° C.-27° C.

Therefore, a cassette is provided, wherein the gelatin or the gelatin based biodegradable material is water-dissolvable gelatin or water-dissolvable gelatin based biodegradable material. Preferably, both the first cassette part and the second cassette part are manufactured from such water-dissolvable gelatin or water-dissolvable gelatin based biodegradable material. It is to be understood that gelatin is a water-dissolvable material and it is to be understood that the gelatin is also a biodegradable material.

It is advantageous when the cassette for the lateral flow assay device and for a lateral flow assay kit comprising such a device, is manufactured in two separate parts: the first cassette part and the second cassette part. A selected test strip can be positioned in/on one of the two cassette parts, either manually, or automatically. After strip positioning, the other cassette part can be connected, again either manually, or automatically, therewith assembling the lateral flow assay device. This provides for example optimal freedom for selecting the appropriate assembly step in a general process of manufacturing test kits.

For a further improvement in ease of production of the lateral flow assay device, when the first and second cassette parts are the lower and upper cassette parts respectively, one of the lower cassette part and upper cassette part of the cassette is formed as an at least substantially flat cover plate that covers one side of the test chamber. The other of the lower cassette part and upper cassette part of the cassette may be formed with a base wall defining an opposite side of the test chamber and with a side wall extending between the base wall and the cover plate around the test chamber. Optionally the base wall and/or side wall comprises at least one opening to enable access to the test chamber from outside the cassette. For example the at least one opening may be an opening through which a biological sample such as a saliva sample can be brought into the test chamber for interaction with the diagnostic test strip. The at least one opening may alternatively or additionally be an opening for inserting the diagnostic test strip or a replacement diagnostic test strip in the test chamber. The at least one opening may also alternatively or additionally be an opening such as a window for visual inspection of at least part of the test chamber. Preferably the base wall and/or side wall comprises a first opening through which a biological sample can be brought into the test chamber and a second separate opening for visual inspection of at least part of the test chamber.

Optionally the first cassette part and second cassette part are made of different materials. However the first cassette part and second cassette part may also be of the same material. It is preferred that the first cassette part and second cassette part are made of the same material. In an aspect the lateral flow assay device has the first cassette part and the second cassette part made of mouldable, preferably injection mouldable and/or 3D printable curable material. Preferably the mouldable and/or 3D printable curable material is gelatin or a biodegradable gelatin composition. Throughout the description and claims, terms “gelatin based (biodegradable) material” and “(biodegradable) gelatin composition” are synonyms having the same meaning. Gelatin is a soluble protein obtained by the partial hydrolysis of collagen derived from the bones, hides and skins of animals such as cows, pigs and fishes. Gelatin is obtained by hydrolyzing collagen using acid, heat, or enzymes, which breaks down the protein into smaller peptides and amino acids that can dissolve in water and form a gel-like substance when cooled. Gelatin is widely used in healthcare and medicine, for example in pill capsules, vaccines and hemostats. Gelatin or gelatin composition is also biodegradable and water dissolvable after gelatin or the gelatin composition has been dried at for example ambient temperature, e.g. dissolvable in water at ambient temperature. The gelatin or gelatin composition may be adapted to be solid at and below normal use temperatures, e.g. below 30° C., and to liquefy when exposed to an increased temperature, e.g. above 40° C. such that it can be easily moulded and/or printed in liquefied form and be cured by drying to ambient air at normal use temperatures such as at room temperature. Advantageously, such moulded or printed gelatin or gelatin composition is dissolvable upon exposure to water for at least half an hour such as at least one hour, for example during emersion in water at 18° C.-25° C. for 30 minutes-4 hours. Herewith, the cassette of the invention is water-dissolvable, and therewith readily biodegradable, when moulded or 3D printed with gelatin material or with a gelatin composition material. Compared to cellulose, gelatin is more readily biodegradable by enzymes due to the lack of crystalline structure, as is present in cellulose, hampering enzymatic decay. Preferably a gelatin composition is used which is adapted to have a hydrophobicity suitable for normal use of the lateral flow assay device. In particular the gelatin composition renders, when cured, the at least one of the first cassette part and second cassette part of the cassette to maintain its form and usability for supporting the diagnostic test strip when exposed under normal conditions to a liquid, e.g. a liquid biological sample. Typical gelatin compositions, such as the gelatin based biodegradable material, suitable for moulding and 3D printing are compositions comprising gelatin and a biodegradable and water soluble plasticizer, such as glycerol, sorbitol and propylene glycol. For moulding and 3D printing gelatin compositions comprising glycerol are preferred. Suitable gelatin compositions, e.g. the gelatin based biodegradable material, for moulding and 3D printing comprise 10-20% glycerol (or sorbitol, or propylene glycol) based on the total weight of the gelatin composition, further comprising 80-90% gelatin based on the total weight of the gelatin composition. Gelatin compositions suitable for application in manufacturing the cassettes according to the invention may comprise 2-50% glycerol based on the total weight of the gelatin composition, the remainder essentially being gelatin. Such biodegradable gelatin compositions are water-dissolvable gelatin compositions, and a cassette manufactured with such a gelatin composition is water dissolvable as here above described, and is biodegradable as here above described. Typically, chemically cross-linked gelatin or chemically cross-linked gelatin compositions are not water-dissolvable. Typically, chemically cross-linked gelatin or chemically cross-linked gelatin compositions are not biodegradable. An example of such a chemically cross-linked gelatin is methacrylated gelatin or UV-cured gelatin hydrogel.

Provided is the cassette, wherein the gelatin based biodegradable material comprises a biodegradable and water-dissolvable plasticizer such as any one or more of glycerol, sorbitol and propylene glycol, preferably glycerol. For example, the gelatin based biodegradable material comprises 2-50% water-dissolvable plasticizer such as glycerol based on the total weight of the material, and for example 50-98% gelatin based on the total weight of the material. Without wishing to be bound by theory, gelatin is reacting as a gelling agent, i.e it is a gel-forming agent when the gelatin is dissolved in a liquid phase such as water, since the colloidal mixture of gelatin and liquid (such as water) forms a weakly cohesive internal structure. Other known gel-forming agents are alginate, pectin, carrageenan, gellan, gelatin, agar, modified starch.

Therefore, a cassette for a lateral flow assay device for in vitro diagnostics of a biological sample comprising a diagnostic test strip inserted into said cassette, the cassette comprising a first cassette part and a second cassette part coupled to each other and defining a test chamber there between containing the diagnostic test strip, wherein the cassette is made of a biodegradable material and wherein at least one of the first cassette part and second cassette part is made of mouldable, preferably injection mouldable and/or 3D printable curable material, wherein the mouldable and/or 3D printable material is gelatin or a gelatin based biodegradable material, is preferred.

Therefore, a lateral flow assay device for in vitro diagnostics of a biological sample comprising a diagnostic test strip inserted into a cassette, the cassette comprising a first cassette part and a second cassette part coupled to each other and defining a test chamber there between containing the diagnostic test strip, wherein the cassette is made of a biodegradable material and wherein at least one of the first cassette part and second cassette part is made of mouldable, preferably injection mouldable and/or 3D printable curable material, wherein the mouldable and/or 3D printable material is gelatin or a gelatin based biodegradable material, is also preferred.

Thus, a cassette for a lateral flow assay device, wherein the first cassette part and the second cassette part are made of the same mouldable and/or 3D printable gelatin or a gelatin based biodegradable material, is preferred.

Thus, a lateral flow assay device, wherein the first cassette part and the second cassette part are made of the same mouldable and/or 3D printable gelatin or a gelatin based biodegradable material, is also preferred.

The cassette can optionally be conveniently formed with a production process in which a warmed sheet of plain plasticized gelatin is placed over a die plate having a number of depression or moulds or numerous die pockets that enable forming the other of the lower cassette part and upper cassette part of the cassette with a base wall and with a side wall. For example by applying vacuum, the warmed sheet is drawn into these depressions or pockets to form corresponding cassette parts. A second sheet of gelatin is carefully placed on top of the formed cassette parts to enable forming of the at least substantially flat cover plate of the cassette. Pressure is then applied by a top plate of the mould to form and seal the cassettes, and the cassettes are cut into individual units.

The mouldable, preferably injection mouldable, and/or 3D printable curable material may alternatively be a plant-based mouldable and/or 3D printable curable material. Preferably the plant-based material is a biodegradable grain-based composition. The biodegradable grain-based composition optionally comprises a wheat flour, preferably a fine or milled flour such as a milled flour from Triticum durum wheat, i.e. Semolina. Plant-based materials are environmental friendly both in use, i.e. have very good biodegradability, and in production. Grain-based materials are moreover available in large quantities and inexpensive, enabling production of low cost cassette parts of the lateral flow assay device. A grain-based composition comprising in particular a wheat flour, preferably a fine or milled flour, provides good moulding and/or 3D printing properties with easy curing after moulding and/or printing, enabling the production of a solid and durable cassette part.

Grain-based compositions thus have several advantages, including:
1. Biodegradability: Similar to gelatin and gelatin compositions, grain-based compositions are biodegradable, i.e. such compositions can be broken down by natural processes and do not accumulate in the environment like traditional fossil-oil based polymer plastics. This makes them a more sustainable and environmentally friendly option.
2. Renewable: Grains such as corn and wheat are renewable resources that can be grown year after year, making them a sustainable choice. Alternatively, and preferred, grain-based compositions are based on by-products or grain processing waste products.
3. Customizability: Grain-based materials can be modified to have different properties, such as strength, flexibility, and biodegradability, making them a versatile choice for a variety of applications.
4. Non-toxic: Grain-based materials are non-toxic, making them safe for use in food and medical applications.
5. Wide availability: Grains are widely available around the world, making them a readily accessible raw material for manufacturing.
   Overall, grain-based compositions offer many benefits over traditional fossil-oil based polymer plastics, including environmental sustainability, cost-effectiveness, and versatility.

Suitable non-animal based materials for (injection) moulding and/or 3D printing of the cassette parts are gelling agents, for example known for their liquid thickening characteristics similar to gelatin and gelatin compositions as here above described: alginate, agar-agar, carrageenan, konjac powder, xantham gum, pectin, gellan, modified starch. For example agar-agar and carrageenan are suitable for injection moulding. However, manufacturing the cassette applying injection moulding using gelatin or a gelatin composition is preferred. Gelatin and therewith also gelatin compositions, such as the gelatin based biodegradable material, are stemming from a bio-based residual product: biodegradable and water dissolvable gelatin. A cassette 3D printed or molded from a gelatin or gelatin composition is dissolvable in water in typically less than 3 hours. This is a major advantage in addition to its biodegradability, in view of discarding cassettes, compared to non-biodegradable cassettes commonly used in clinical practice which are made of fossil oil-based polymer plastics.

Overall, the gelatin, gelatin compositions (e.g. gelatin based biodegradable material), grain-based materials and further non-animal based materials as hereabove described, have several advantages when moulded or 3D printed into the cassettes according to the invention, when compared to certain cassettes made of cellulose. Typically, the cassettes made of the biodegradable material according to the current invention are smaller in size than cellulose based cassettes for lateral flow assays. This makes the cassettes, for example a gelatin or gelatin composition based cassette, more suitable since the lateral flow assay device has a smaller size, lowering volume required for stock piling and transportation, lowering overall demand for natural resources, etc. In addition, a cassette for lateral flow assay devices according to the invention is more robust and more resistant to e.g. cracking, for example when these parameters are compared between a cassette made of gelatin composition according to the invention and a cassette made of cellulose.

Optionally, the cassette and the lateral flow assay device have at least one of the first cassette part, i.e. the lower part or the inner part, and second cassette part, i.e. the upper part or the outer part, moulded, preferably injection moulded or 3D printed in one piece. Preferably at least the other of the first cassette part and second cassette part with base wall and side wall is moulded or 3D printed. The first cassette part and the second cassette part may each be (injection) moulded or 3D printed in one piece. The one of the first cassette part and second cassette part of the cassette, particularly when formed as an at least substantially flat cover plate, may also be made in another way, such as using a dipping technique.

Dipping is a coating method in which an object is dipped into a liquid coating material, such as liquid gelatin or liquid gelatin based biodegradable material, and then removed to allow the excess coating to drip off. The coated object is then allowed to dry or may be cured by for example heating. The dipping method is used in industrial manufacturing processes for coating a small to medium-sized object (e.g. a stamp for manufacturing the cassette or two stamps for manufacturing the two cassette parts, of the lateral flow assay device, by dipping the stamp(s) in e.g. the gelatin or the gelatin based biodegradable material) with a uniform layer of material, e.g. the gelatin or the gelatin based biodegradable material. Thus, the dipping method is suitable for providing e.g. a gelatin coating on a substrate. An example of providing a substrate (e.g. a stamp for a cassette part) is:
1.Gelatin is dissolved in hot water or another solvent to provide liquid solution of gelatin.
2.The object or substrate to be coated is provided (the object or substrate is optionally pretreated with a primer and/or first provided with a different coating, before the gelatin is coated).
3.The object or substrate is then dipped into the gelatin solution, ensuring that the entire surface is covered with the liquid gelatin.
4.The object or substrate is then slowly lifted out of the gelatin solution, allowing excess gelatin to drip off.
5.The coated object or substrate is then allowed to dry and/or for example cured by for example heating.

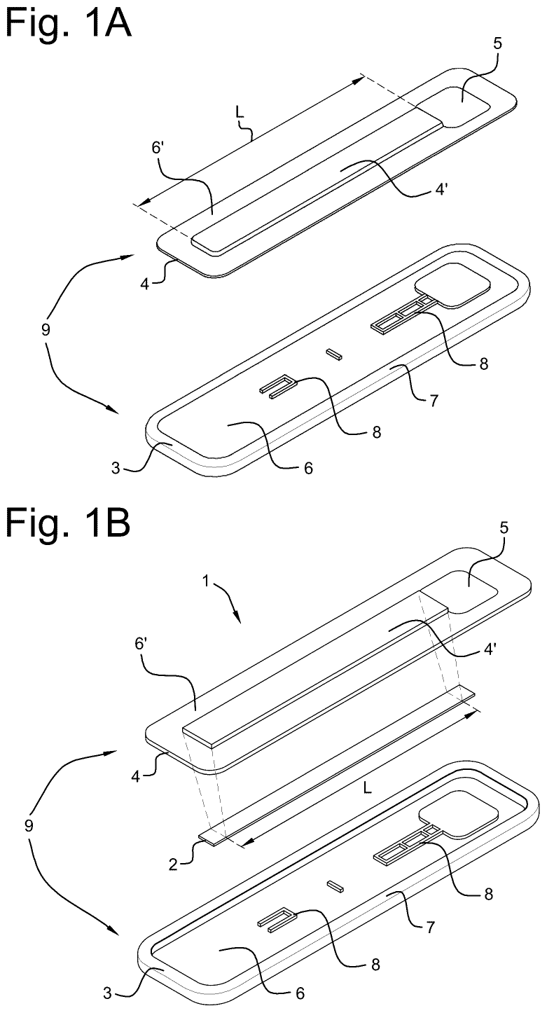

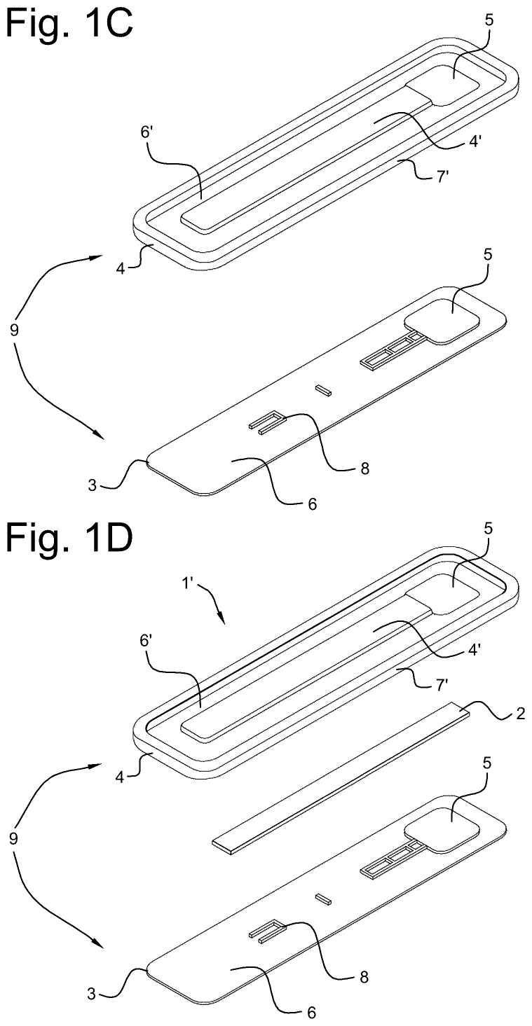

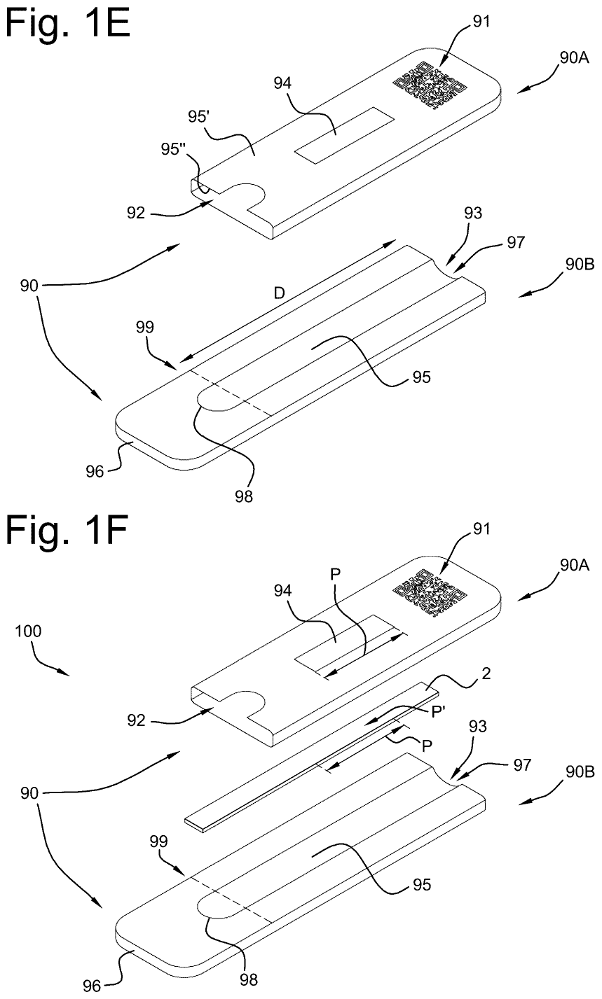

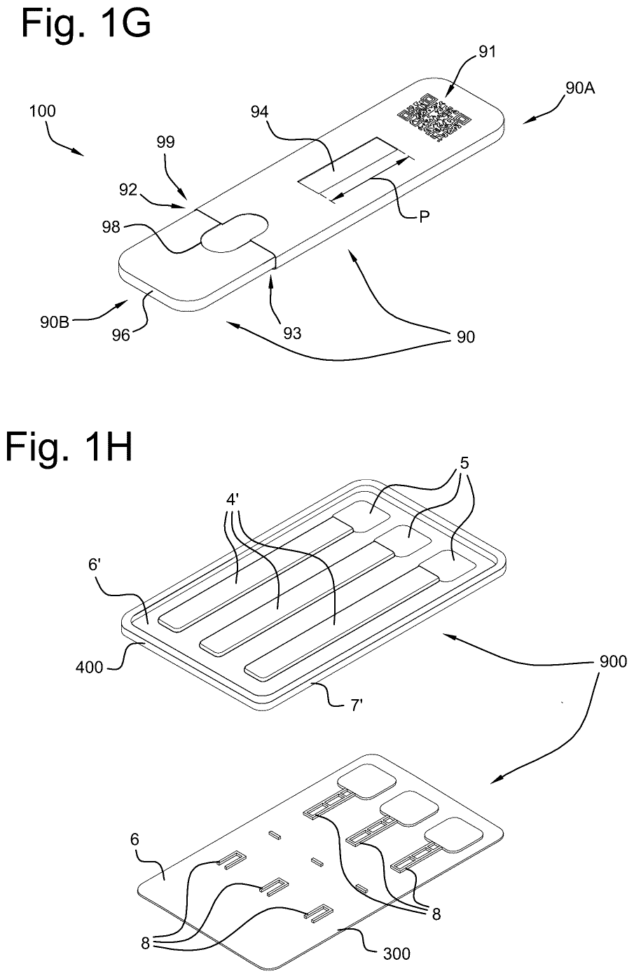

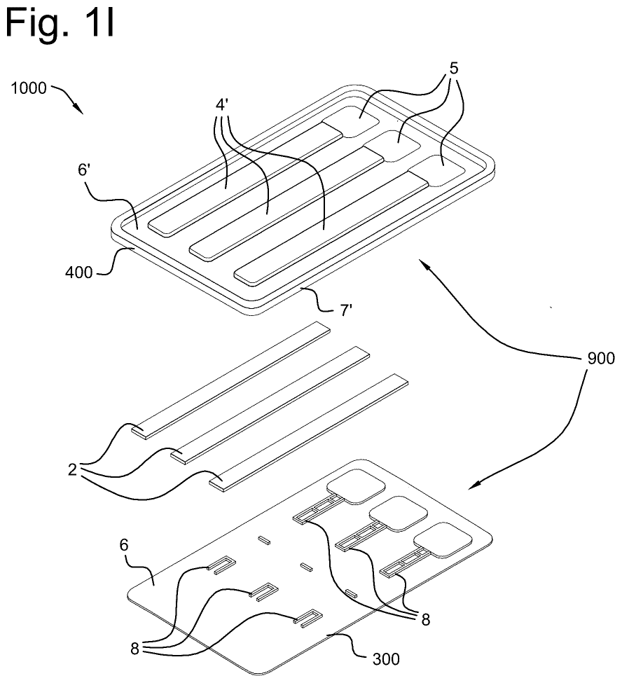

The dipping method can be used to create a thin, uniform layer of e.g. gelatin coating on the object or substrate. The thickness of the coating can be controlled by adjusting the concentration of the gelatin in the gelatin solution and by selecting the number of dips, such as selecting 1-5 dips. The dipping method can also be applied to provide multi-layer coatings by repeating the dipping and drying process. An example of a cassette consisting of two cassette parts is shown in FIG. 1E, which cassette parts can be manufactured by applying the here-above outlined dipping method. Such dipping method is applicable for mass production of the (two) cassette parts of the cassette. For example, after manufacturing the two cassette parts, a diagnostic test strip can be inserted, therewith providing the cassette with test strip for a lateral flow assay device (refer to FIG. 1E, F).

In an aspect of the cassette or of the lateral flow assay device comprising the cassette, the first cassette part and the second cassette part are coupled to each other with a manually releasable connection. The first cassette part and second cassette part can accordingly be manually released from each other to enable inspection of the test chamber and/or to exchange the at least one, such as one, diagnostic test strip for replacement diagnostic test strip(s) if desired. For instance the manually releasable connection can be realized with unlockable snap-fit connection means that may be moulded, preferably injection molded, or 3D printed integrally with at least one of the first cassette part and second cassette part. Another advantage of including a releasable connection between the first and second parts of the cassette is the provision of the option to easily and readily remove the test strip from the lateral flow assay device, before the biodegradable two parts of the cassettes are disposed for biodegradation or for example for dissolution in water (e.g. when the cassette is moulded from gelatin or a biodegradable gelatin composition).

For a cassette according to the invention, the first cassette part is a lower cassette part and the second cassette part is an opposite upper cassette part configured to be coupled to each other such that, after coupling, at least one test chamber between the lower cassette part and the upper cassette part is defined, said at least one test chamber configured to contain the at least one diagnostic test strip.

An alternative embodiment is the cassette according to the invention, wherein the first cassette part is an inner cassette part and the second cassette part is an outer cassette part configured to slidably receive the inner cassette part, the inner cassette part and the outer cassette part configured to be slidably coupled to each other such that, after coupling, at least one test chamber between the inner cassette part and the outer cassette part is defined, said at least one test chamber configured to contain the at least one diagnostic test strip.

A multiplex lateral flow assay is a type of diagnostic test that allows for the detection of multiple analytes in a single sample. This is achieved by incorporating multiple test lines on a single strip, each line designed to capture a different analyte of interest. Alternatively, this is achieved by applying multiple test strips in a single lateral flow assay device. That is to say, according to the invention, a cassette is provided which comprises more than one test strip chamber such as two, three, four, five, six, seven, eight, nine, ten, eleven or twelve test strip chambers each configured to receive a separate test strip. The sample is applied to the strip or to two or more of the separate strips, and then migrates through the strip(s), and when it encounters the test lines, any captured analytes will cause the line to become visible, indicating a positive result. The use of multiple test lines or the use of multiple test strips in parallel in a single lateral flow assay device, allows for simultaneous detection of multiple analytes, which can save time and resources in comparison to running separate tests for each analyte. Multiplex lateral flow assays have applications in various fields, including medical diagnostics, environmental monitoring, and food safety testing.

One of the main advantages of multiplex lateral flow assays is that multiple analytes simultaneously can be tested, reducing the need for multiple assays and thereby reducing waste.

Cassettes used for multiplex lateral flow assay devices are generally larger compared to cassettes applied in a lateral flow assay device that comprises a single test strip, since several test strips need to fit in the cassette. Therefore, provision of a biodegradable cassette according to the invention for a multiplex lateral flow assay device, provides for a more sustainable solution (less waste, ease of biodegradation, for gelatin-based and gelatin composition-based cassettes further accompanied with the benefit of ease of disposal by dissolution in water, e.g. at ambient temperature).

The invention provides cassettes for a multiplex lateral flow assay comprising at least two diagnostic test strips, preferably two, three, four, five, six, seven, eight, nine, ten, eleven or twelve test strips, more preferable two, three or four test strips, the test strips inserted into said cassette, the cassette comprising a first cassette part and a second cassette part configured to be coupled to each other and defining after coupling at least two test chambers there between configured to contain the at least two diagnostic test strips.

In another aspect herein there is provided a lateral flow assay kit comprising a lateral flow assay device as described herein and at least one of the group consisting of a replacement diagnostic test strip, a sample taker such as a swab, a sample holder such as a tube, and a waste container such as a zip-lock bag. Preferably, in addition to the lateral flow assay device also the at least one of the group consisting of a replacement diagnostic test strip, a sample taker such as a swab, a sample holder such as a tube, and a waste container such as a zip-lock bag is biodegradable and/or made of 3D printable curable material.

Thus, provided is a lateral flow assay kit comprising a lateral flow assay device comprising the cassette according to the invention and at least one diagnostic test strip inserted into said cassette, and at least one of the group consisting of at least one replacement diagnostic test strip, a sample taker such as a swab, a sample holder such as a tube, and a waste container such as a zip-lock bag, wherein optionally at least one of the at least one diagnostic test strip and the group consisting of at least one replacement diagnostic test strip, a sample taker such as a swab, a sample holder such as a tube, and a waste container such as a zip-lock bag is made of a biodegradable material.

Preferably, apart from the cassette, also at least the diagnostic test strip and replacement diagnostic test strip are biodegradable.

An aspect of the invention relates to a method of manufacturing the cassette according to the invention, comprising or consisting of the steps:
(i) providing molten gelatin or the gelatin based biodegradable material;
(ii) providing the first cassette part and the second cassette part by moulding, preferably injection moulding of the molten gelatin or the gelatin based biodegradable material of step (i); optionally
(iii) coupling the first cassette part and the second cassette part provided in step (ii), such that at least one test chamber there between is defined configured to contain at least one diagnostic test strip; and optionally
(iv) providing, for example by printing, an exposed outer surface (95′) of the second cassette part (4, 90B, 400) with a mark, text, figure, label and/or code (91), such as a QR code,
   therewith providing the cassette.

A QR code could be printed on one of the two cassette parts, for example for the purpose of data storage or in order to share personalised results. Alternatively or in addition a copyright sign, label, number, etc. can be printed or stamped onto the outer surface of the cassette.

The method can also suitably applied when any other of the biodegradable non-animal based mouldable and/or 3D printable materials are used for manufacturing the cassette of the invention. For example, the method is applied with grain-based material, carrageenan or agar-agar, however application in the method of gelatin or gelatin composition, i.e. the gelatin based biodegradable material, is preferred. These and other objects and aspects are hereinafter further elucidated by the appended drawings and the corresponding embodiments, which forms part of the present application. The drawings are not in any way meant to reflect a limitation of the scope of the invention, unless this is clearly and explicitly indicated. Thus, while the invention has been described in terms of several embodiments, it is contemplated that alternatives, modifications, permutations and equivalents thereof will become apparent to one having ordinary skill in the art upon reading the specification and upon study of the drawings. Changes can be made without departing from the scope which is defined by the appended claims. The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The embodiments of the invention described herein can operate in combination and cooperation, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a cassette comprising part A and part B” should not be limited to a cassette consisting only of parts A and B, rather with respect to the present invention, the only enumerated components of the cassette are part A and part B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

### Embodiments

An aspect of the invention is a lateral flow assay device ‘LFAD’ for in vitro diagnostics of a biological sample comprising a diagnostic test strip inserted into a cassette, the cassette comprising a lower cassette part and an opposite upper cassette part coupled to each other and defining a test chamber there between containing the diagnostic test strip, wherein the cassette is made of a biodegradable material and wherein at least one of the lower cassette part and upper cassette part is made of 3D printable curable material.

An embodiment is the LFAD as hereabove described, wherein the lower cassette part and the upper cassette part are made of 3D printable material.

An embodiment is the LFAD as hereabove described, wherein the 3D printable material is gelatin or a gelatin based biodegradable material.

An embodiment is the LFAD as hereabove described, wherein the 3D printable material is a plant-based 3D printable curable material, preferably a plant-based material comprising a biodegradable grain-based composition.

An embodiment is the LFAD as hereabove described, wherein the lower cassette part and the upper cassette part are made of the same 3D printable material.

An embodiment is the LFAD as hereabove described, wherein at least one of the lower cassette part and upper cassette part is 3D printed in one piece.

An embodiment is the LFAD as hereabove described, wherein the lower cassette part and the upper cassette part are each 3D printed in one piece.

An embodiment is the LFAD as hereabove described, wherein the lower cassette part and the upper cassette part are coupled to each other with a manually releasable connection. An aspect of the invention is a lateral flow assay kit comprising an LFAD according to any one of the foregoing aspect and embodiments and at least one of the group consisting of a replacement diagnostic test strip, a sample taker such as a swab, a sample holder such as a tube, and a waste container such as a zip-lock bag.

### EXAMPLE

### Moulding of a Cassette With Gelatin Based Biodegradable Material

For manufacturing of a two-part cassette with for example a lower part and an opposite upper part (see for example FIG. 1B, 1D), from biodegradable gelatin composition comprising gelatin and glycerol (a gelatin based biodegradable (and water-dissolvable) material), the following steps were applied:
1. Mixing: First, provided dried gelatin composition granules, obtained from gelatin composition comprising glycerol and gelatin, were mixed with water in a heated tank to form a solution.
2. Moulding: The gelatin solution is then poured into the two moulds, which are designed to form the shapes of the two parts of the cassette. The moulds were made of plastic.
3. Cooling: Once the gelatin solution was poured into the moulds, it was allowed to cool down to room temperature and therewith to solidify. Alternatively, the cooling process was accelerated by placing the moulds after moulding at a temperature of about 4° C.
4. Drying: After cooling, the gelatin test cassette parts were removed from the moulds and dried to remove any excess moisture. Drying was established by placing the moulded cassette parts in a low-humidity environment at room temperature.
5. Assembly: Finally, components of a regular lateral flow assay device were assembled together, including the two parts of the cassette, a diagnostic test strip, a sample pad, a conjugate pad, a nitrocellulose membrane, and an absorbent pad.

### DESCRIPTION OF EMBODIMENTS

In the drawings:

FIG. 1A and 1B show an exploded view of an embodiment of a cassette 9 and a lateral flow assay device 1 comprising said cassette 9 in accordance with a first aspect herein.

The description of the embodiment is not limited to the example shown in the figures and the reference numbers used in the detailed description and the claims are not intended to limit the description of the embodiment, but are included to elucidate the embodiment by referring to the example shown in the figures.

In the embodiment as shown in FIG. 1B, the lateral flow assay device 1 for in vitro diagnostics of a biological sample comprises a diagnostic test strip 2 inserted into a cassette 9 (see FIG. 1A), the cassette 9 (FIG. 1A and 1B) comprising a lower cassette part 3 and an opposite upper cassette part 4. The lower cassette part 3 and the opposite upper cassette part 4 are coupled to each other and define a test chamber containing the diagnostic test strip 2. The upper cassette part 4 is formed as an at least substantially flat cover plate that covers one side of the test chamber. The upper cassette part 4 has an opening 5 that forms an entrance through which a biological sample can be brought into the test chamber for interaction with the diagnostic test strip 2. The upper cassette part 4 is transparent at least in part, e.g. upper cassette part section 4′, in order for a user to be able to view at least a part of the diagnostic test strip 2 that has the indicator means for the predetermined test compound of the lateral flow assay device 1 without having to open the lateral flow assay device 1.

The lower cassette part 3 is formed with a base wall 6 defining an opposite side of the test chamber and with a side wall 7 extending between the base wall 6 and the upper cassette part 4 around the test chamber. The lower cassette part 3 has on the base wall 6 support means 8 for supporting the diagnostic test strip 2 at a distance above the base wall 6 in a correct position for reliable interaction in use with a biological sample.

FIG. 1C and 1D show an exploded view of a second embodiment of a cassette 9 and a lateral flow assay device 1′ comprising said cassette 9 in accordance with the first aspect herein. This second embodiment is basically the same as the hereabove outlined embodiment displayed in FIG. 1A and 1B, except for the location of the side wall 7′. In the embodiment displayed in FIG. 1A, the side wall 7 is comprised by the lower cassette part 3. In the embodiment displayed in FIG. 1C and 1D, the equivalent side wall 7′ is comprised by the upper cassette part 4, and is extending between the base wall 6′ of the upper cassette part and the lower cassette part 3 around the test chamber.

FIG. 1E and 1F show an exploded view of an embodiment of a cassette 90 and a lateral flow assay device 100 comprising said cassette 90 in accordance with a further aspect herein. FIG. 1G shows cassette 90.

In the embodiment as shown in FIG. 1F and 1G, the lateral flow assay device 100 for in vitro diagnostics of a biological sample comprises a diagnostic test strip 2 inserted into a cassette 90 (see FIG. 1E), the cassette 90 (FIG. 1E, 1F and 1G) comprising an inner cassette part 90B and an outer cassette part 90A configured to slidably receive at least a portion of the inner cassette part 90B through opening 92 of the outer cassette part 90A. Inner cassette part 90B is slidable into outer cassette part 90A from the proximal end 93 up to the distal end 96 of the inner cassette part 90B, or inner cassette part 90B is slidable into outer cassette part 90A from the proximal end 93 up to a portion with length D of the inner cassette part 90B, D elongating from the proximal end 93 to inner-cassette part/outer-cassette part connecting point 99. Inner cassette part 90B comprises a test-strip receiving area 95, forming one side of a test chamber when the inner cassette part 90B is engaged with outer cassette part 90A (FIG. 1G). The outer cassette part 90A is formed as an at least substantially hollow container with an opening 92 and with an upper wall 95′, of which the inner side inside the outer cassette part 90A covers one side of the test chamber. Thus, when the inner cassette part 90B is slided (partially) into outer cassette part 90A, the inner cassette part 90B and the outer cassette part 90A are coupled to each other and define a test chamber containing the diagnostic test strip 2.

The outer cassette part 90A is transparent at least in part, e.g. outer cassette part section 94, in order for a user to be able to view at least a part of the diagnostic test strip 2 that has the indicator means for the predetermined test compound of the lateral flow assay device 100 without having to open the lateral flow assay device 100.

Upper wall 95′ comprises a printed QR code 91 on the outer surface of outer cassette part 90A.

In FIG. 1G, inner cassette part 90B is slided into outer cassette part 90A, from its proximal end 93 (being the proximal end 97 of the test-strip receiving area 95) up to inner-cassette part/outer-cassette part connecting point 99 near the distal end 98 of the test-strip receiving area 95. Portion P′ with length P of an inserted test strip 2, received by the test-strip receiving area 95 and now embedded in the test chamber, is visible through transparent outer cassette part section 94 with length P.

FIG. 1H and 1I show an exploded view of a third embodiment of a cassette 900 and a lateral flow assay device 1000 comprising said cassette 900 in accordance with the first aspect herein. This third embodiment is basically the same as the hereabove outlined embodiment displayed in FIG. 1C and 1D, except for the number of test chambers defined when the lower cassette part 3 (FIG. 1C) or 300 (FIG. 1H) and the opposite upper cassette part 4 (FIG. 1C) or 400 (FIG. 1H) are coupled to each other and define a single test chamber containing the diagnostic test strip 2 for cassette 9 or define three test chambers for receiving three diagnostics test strips 2 for cassette 900.

The lower cassette part 3, 300 is in an embodiment moulded in one piece using injection moulding or is in an embodiment 3D printed in one piece, using a gelatin composition, such as a gelatin based biodegradable material, e.g. a gelatin based biodegradable and water-dissolvable material. The upper cassette part 4, 400 is also in an embodiment moulded in one piece using injection moulding or is in an embodiment 3D printed in one piece, using the same gelatin composition. The gelatin based biodegradable material is adapted to be solid at normal use temperatures and conditions, such that after moulding or printing and cooling to approximately room temperature of the test cassette parts the cassette is solid and durable for use. The gelatin based biodegradable material is further adapted to have a transparency in solid form that enables the user to visually inspect the test chamber through the upper cassette part. The gelatin based biodegradable material is fully biodegradable, so that the lateral flow assay device is plastic free and environmental friendly.

While the current application may describe features as part of the same embodiment or as parts of separate embodiments, the scope of the present invention also includes embodiments comprising any combination of all or some of the features described herein.

1. A cassette suitable for application in a lateral flow assay device for in vitro diagnostics of a biological sample, said lateral flow assay device comprising at least one diagnostic test strip, preferably one, two, three or four diagnostic test strip(s), inserted into said cassette, the cassette comprising a first cassette part and a second cassette part configured to be coupled to each other and defining after coupling at least one test chamber there between configured to contain the at least one diagnostic test strip, wherein the cassette is made of a biodegradable material and wherein at least one of the first cassette part and second cassette part is made of mouldable material, wherein the mouldable material is gelatin or a gelatin based biodegradable material.

2. The cassette according to claim 1, wherein the gelatin or the gelatin based biodegradable material is water-dissolvable gelatin or water-dissolvable gelatin based biodegradable material.

3. The cassette according to claim 1, wherein the gelatin based biodegradable material comprises a biodegradable and water-dissolvable plasticizer such as any one or more of glycerol, sorbitol and propylene glycol, preferably glycerol.

4. The cassette according to claim 1, wherein the first cassette part and the second cassette part are made of mouldable, preferably injection mouldable gelatin or a gelatin based biodegradable material.

5. The cassette according to claim 1, wherein the first cassette part and the second cassette part are made of the same mouldable, preferably injection mouldable gelatin or a gelatin based biodegradable material.

6. The cassette according to claim 1, wherein at least one of the first cassette part and second cassette part is moulded, preferably injection moulded in one piece.

7. The cassette according to claim 6, wherein the first cassette part and the second cassette part are each moulded, preferably injection moulded in one piece.

8. The cassette according to claim 1, wherein the first cassette part and the second cassette part are configured to be coupled to each other with a manually releasable connection.

9. The cassette according to claim 1, wherein the first cassette part is a lower cassette part and the second cassette part is an opposite upper cassette part configured to be coupled to each other such that, after coupling, at least one test chamber between the lower cassette part and the upper cassette part is defined, said at least one test chamber configured to contain the at least one diagnostic test strip.

10. The cassette according to claim 1, wherein the first cassette part is an inner cassette part and the second cassette part is an outer cassette part configured to slidably receive the inner cassette part, the inner cassette part and the outer cassette part configured to be slidably coupled to each other such that, after coupling, at least one test chamber between the inner cassette part and the outer cassette part is defined, said at least one test chamber configured to contain the at least one diagnostic test strip.

11. A lateral flow assay kit comprising a lateral flow assay device comprising the cassette according to claim 1 and at least one diagnostic test strip inserted into said cassette, and at least one of the group consisting of at least one replacement diagnostic test strip, a sample taker such as a swab, a sample holder such as a tube, and a waste container such as a zip-lock bag, wherein optionally at least one of the at least one diagnostic test strip and the group consisting of at least one replacement diagnostic test strip, a sample taker such as a swab, a sample holder such as a tube, and a waste container such as a zip-lock bag is made of a biodegradable material.

12. A method of manufacturing the cassette according to claim 1, comprising or consisting of the steps:
(i) providing molten gelatin or the gelatin based biodegradable material;
(ii) providing the first cassette part and the second cassette part by moulding, preferably injection moulding of the molten gelatin or the gelatin based biodegradable material of step (i); optionally
(iii) coupling the first cassette part and the second cassette part provided in step (ii), such that at least one test chamber there between is defined configured to contain at least one diagnostic test strip; and optionally
(iv) providing, for example by printing, an exposed outer surface of the second cassette part with a mark, text, figure, label and/or code, such as a QR code, therewith providing the cassette.
